(19) United States
(12) Patent Application Publication
(10) Pub. No.: US 2025/0177011 A1
(43) Pub. Date: Jun. 5, 2025

(54) BONE STABILIZING IMPLANTS AND METHODS OF PLACEMENT ACROSS SI JOINTS

(71) Applicant: SI-Bone Inc., Santa Clara, CA (US)

(72) Inventors: Mary E. STUART, Santa Clara, CA (US); Bret W. SCHNEIDER, San Jose, CA (US); Francois FOLLINI, Austin, TX (US); Craig S. BARTLETT, Shelburne, VT (US); Bharat M. DESAI, Golden, CO (US); Michael J. GARDNER, Menlo Park, CA (US); GianLuigi MORO, Rodano (IT); Sven H. VAN HELDEN, Hattem (NL); Kyle L. NISHKIAN, San Francisco, CA (US); David W. POLLY, Edina, MN (US); Jed S. VANICHKACHORN, Richmond, VA (US); Andy J. KRANENBURG, Central Point, OR (US); Sharad S. RAJPAL, Golden, CO (US); Nikolai G. RAINOV, Augsburg (DE)

(21) Appl. No.: 18/977,789

(22) Filed: Dec. 11, 2024

Related U.S. Application Data
(63) Continuation of application No. 18/309,686, filed on Apr. 28, 2023, now Pat. No. 12,201,330.
Application No. 18/309,686 is a continuation of application No. 17/104,753, filed on Nov. 25, 2020, now Pat. No. 11,672,570.
(60) Provisional application No. 62/941,507, filed on Nov. 27, 2019.

(51) Int. Cl. A61B 17/70; A61B 17/86; A61F 2/30; B33Y 80/00

(52) U.S. Cl. CPC A61B 17/7055; A61B 17/864; A61F 2/30988; B33Y 80/00; A61F 2002/30622; A61F 2002/30784; A61F 2002/30858; A61F 2002/30985; A61F 2002/30995

(57) ABSTRACT

Threaded sacro-iliac joint stabilization (e.g., fusion, fixation) implants and methods of implantation and manufacture. Some implants include a threaded distal region, an optionally threaded central region, and an optionally threaded proximal region. The distal, central, and proximal regions have lengths such that when the implant is laterally implanted across a SI joint, the distal region can be positioned in a sacrum, the central region can be positioned across an SI-joint, and the proximal region can be positioned in an ilium.

### CROSS REFERENCE TO RELATED APPLICATIONS

This application is a continuation of U.S. patent application Ser. No. 18/309,686, filed Apr. 28, 2023, which is a continuation of U.S. patent application Ser. No. 17/104,753, filed Nov. 25, 2020, now U.S. Pat. No. 11,672,570, which claims the benefit of U.S. Provisional Application No. 62/941,507, filed Nov. 27, 2019, the entire disclosures of which are incorporated by reference herein in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

The SI-Joint functions in the transmission of forces from the spine to the lower extremities, and vice-versa. The SI-Joint has been described as a pain generator for up to 22% of lower back pain. To relieve pain generated from the SI Joint, SI Joint fusion is typically indicated as surgical treatment, e.g., for degenerative sacroiliitis, inflammatory sacroiliitis, iatrogenic instability of the sacroiliac joint, osteitis condensans ilii, or traumatic fracture dislocation of the pelvis. There is a continued need for improved threaded SI Joint fixation and fusion implants.

### SUMMARY OF THE DISCLOSURE

One aspect of the disclosure is a threaded bone implant (“implant”). The implant may include an elongate body having a distal end and a proximal end. The elongate body may include a threaded multi-lead distal region, a threaded single-lead central region disposed proximally of the multi-lead distal region, and a threaded multi-lead proximal region disposed proximally of the single-lead central region. The elongate body has a length, and the threaded multi-lead distal region, the threaded single-lead central region, and the threaded multi-lead proximal region may each have individual lengths such that when the implant is laterally implanted, the multi-threaded distal region can be positioned in a sacrum, the single-lead central region can be positioned across a SI-joint, and the multi-lead proximal region can be positioned in an ilium.

In this aspect, the threaded multi-lead distal region may be better adapted to anchor into dense sacral bone than the threaded single lead central region.

In this aspect, the threaded multi-lead proximal region may be better configured to anchor into dense iliac bone than the threaded single lead central region.

In this aspect, one or both of the multi-lead distal region and the multi-lead proximal region may be a dual-lead threaded region.

In this aspect, the multi-lead distal region, single-lead central region, and multi-lead proximal region may all comprise an inner shank from which the respective thread radially extends and a porous network of interconnected struts disposed about the inner shank and in between threads. A proximal region or portion of the threaded single-lead central region may be free of a porous network of interconnected struts, and a threaded single-lead central region may have a major diameter from 9 mm to 11 mm (the outer diameter of the thread).

In this aspect, a threaded multi-lead distal region may be a dual-lead distal region comprising a pattern of high and low threads.

In this aspect, a threaded multi-lead proximal region may be a dual-lead distal region comprising a pattern of high and low threads.

In this aspect, a first thread may be continuous and extend from the distal region, through the central region, and into the proximal region. A continuous thread in this regard may be interrupted by a plurality of fenestrations and/or flutes extending through the elongate body.

In this aspect, the elongate body may further have a plurality of helical flutes formed therein, each of the plurality of flutes extending in the multi-lead distal region, the single-lead central region, and optionally in the multi-lead proximal region. A plurality of helical flutes may consist of three helical flutes in the elongate implant body. Each of a plurality of flutes may have a plurality of fenestrations aligned with the respective flute, the fenestrations spaced from each other along a length of the flute and extending into an elongate body central lumen or area. Each of a plurality of fenestrations may have a radially inward tapered configuration. At least one of a plurality of fenestrations may be disposed in the distal region, at least one of the plurality of fenestrations may be disposed in the central region, and at least one of the plurality of fenestrations may be disposed in the proximal region. In some embodiments the proximal region is free of fenestrations.

In this aspect, a first fenestration in the distal region may be larger than a second fenestration in the central region, and optionally each of a plurality of distal fenestrations may be larger than each of a plurality of central fenestrations.

In this aspect, the elongate body may further comprise a plurality of fenestrations therethrough. A first fenestration in the distal region may be larger than a second fenestration in the central region. In some embodiments, each of a plurality of distal fenestrations may be larger than each of a plurality of central fenestrations.

In this aspect, a proximal region of the elongate body may be tapered, with a proximal end having a larger radial dimension than a distal end of the proximal region.

In this aspect, at least one of the threads may have an inverse fillet that is curved.

In this aspect, a proximal end of the elongate body may be counter-sunk.

In this aspect, the length of the distal region may be from 10 mm to 22 mm.

In this aspect, the length of the central region may be from 8 mm to 56 mm.

In this aspect, the length of the proximal region may be from 6 mm to 10 mm.

This aspect may further include any suitable implant feature described herein.

One aspect of this disclosure is a threaded bone stabilization implant adapted for a lateral delivery and sized for placement across a sacro-iliac (“SI”) joint. The implant includes an elongate body having a distal end and a proximal end. The elongate body may include a threaded distal region, a threaded central region disposed proximally of the distal region, and a proximal region disposed proximally of the central region. The elongate body may further include a plurality of helical flutes, each of the plurality of helical flutes having formed therethrough a plurality of fenestrations extending into a central lumen. The body may have a length, and the threaded distal region, the threaded central region, and the proximal region may each have individual lengths such that when the implant is laterally implanted, the threaded distal region can be positioned in a sacrum, the threaded central region can be positioned across an SI-joint, and the proximal region can be positioned in an ilium.

This aspect may additionally comprise any other suitable threaded implant feature described herein.

One aspect of this disclosure is a threaded bone implant. The implant includes an elongate body extending from a distal end to a proximal end. The elongate body may include one or more helical threads, each of the one or more helical threads extending axially along at least a portion of the elongate body. The elongate body may include an inner shank or inner member from which the one or more helical threads radially extend. The elongate body may also include a porous network of interconnected struts disposed about the inner shank (or inner member) and about a longitudinal axis of the elongate bone implant body. A porous network of interconnected struts may be disposed between the one or more helical threads along at least a section of the elongate body, and optionally disposed in each of a distal region, a central region, and a proximal region of the implant. In some examples a porous network of interconnected struts has a continuous helical configuration through a distal region, a central region, and into a proximal region. Continuous in this context and as used herein includes discontinuities in the porous network due to one or more flutes and/or one or more fenestrations. A porous network of interconnected struts may have an outer dimension that is less than a major diameter of the one or more helical threads.

This aspect may include any other suitable threaded implant feature described herein.

One aspect of this disclosure is a method of manufacturing a threaded bone implant. The method may include printing a threaded bone implant from a distal end to a proximal end (although printing from a proximal end (head) to a distal end (tip) may be used in some alternative embodiments). Printing the implant may include printing an inner shank, printing one or more helical threads extending radially from the inner shank, each of the one or more helical threads extending along at least a portion of the threaded bone implant. The method may include printing a porous network of interconnected struts about the inner shank, about a long axis of the elongate bone implant body, and between at least a section of the one or more helical threads. The porous network of interconnected struts generally has an outer dimension less than a major diameter of the one or more helical threads.

This aspect may include any other suitable method step herein, and may be a computer executable method stored in a memory and adapted to be executed by a processor or processing component, concepts of which are known (e.g., one or more pieces of software, an algorithm, etc.)

One aspect of the disclosure is a method of 3D printing a threaded bone implant. The method may include printing a threaded bone implant from a distal end to a proximal end. Printing the implant may include printing a sacrificial distal tip, printing a threaded bone implant above the sacrificial distal tip, and subsequent in time to printing the threaded bone implant, removing the sacrificial tip and forming a distal end on the threaded bone implant.

This aspect may include any other suitable method described herein.

One aspect of this disclosure is a 3D printed threaded bone implant. The implant may include a 3D printed implant body having a distal end and a proximal end. The implant body may have one or more threads thereon extending radially outward from an inner shank. At least one thread may form an angle greater than 45 degrees relative to a long axis of the implant body.

This aspect may include any other suitable feature related to threaded bone implants herein.

### BRIEF DESCRIPTION OF THE DRAWINGS

FIG. 1 is a side view of an exemplary threaded implant.

FIG. 2 is a side view of an exemplary threaded implant.

FIG. 3 is a side view of an exemplary threaded implant.

FIG. 4 is a side view of an exemplary threaded implant.

FIG. 5 is a side view of an exemplary lag threaded implant and washer.

FIG. 6 is a side view of an exemplary lag threaded implant and washer.

FIG. 7 is a perspective view of an exemplary threaded implant.

FIG. 8 is a side view of an exemplary threaded implant including a fluted region, a fenestration, and a porous network of interconnected struts.

FIG. 9 is a side view of an exemplary threaded implant including a porous network of interconnected struts between threads.

FIG. 10 is a side view of an exemplary threaded implant including flutes, as well as a porous network of interconnected struts disposed between threads.

FIG. 11 illustrates an exemplary thread angle alpha referenced herein.

FIGS. 12A and 12B illustrates an exemplary orientation for manufacturing threaded implants herein.

### DETAILED DESCRIPTION

The disclosure relates generally to threaded bone stabilizing implants, which may be used for fixation and/or fusion, for example. The bone stabilizing implants described herein are generally sized and configured to be delivered in a lateral delivery pathway and implanted such that a distal region of the implant is implanted in a sacrum, an intermediate region is implanted in or across a sacro-iliac (“SI”) joint, and a proximal region is implanted in an ilium. The bone implants herein include one or more threads along at least a portion of the implant, which allows the implants to be rotated into and anchored into bone during implantation. When an implant herein is referred to as a threaded implant, it refers to an implant having one or more threads, any one of which may extend along at least a portion of a length of the implant.

The threaded bone implants herein generally include different regions or portions along their lengths that are sized and/or configured to provide functionality based at least partially on the anatomical region in which they implanted. For example, implants herein may have distal regions that are sized (e.g., length and/or width) and configured (e.g., threaded) such that the distal region is adapted with functionality to anchor into relatively more dense cancellous sacral bone. The functionality may be compared relative to other regions of the implant that are not so sized and/or configured, or to other types of implants that are not so sized and/or configured in the manner(s) described herein.

The disclosure herein may be related to disclosure from U.S. Publication. Nos. 2018/0228621, 2013/0296953 and 20150105828, the entire disclosures of which are incorporated by reference herein for all purposes.

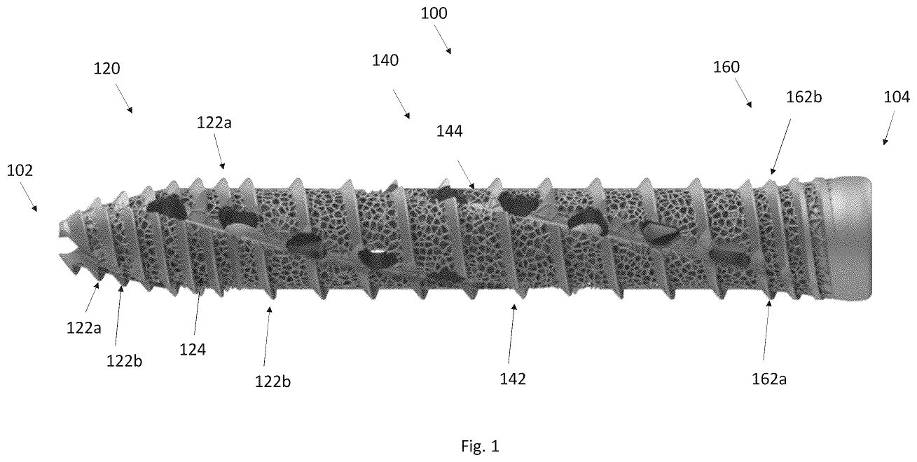

FIG. 1 illustrates an exemplary bone stabilization implant 100 that includes an elongate body as shown, the elongate body extending from distal end 102 to proximal end 104. The elongate body includes distal anchor region 120, intermediate or central region 140, and proximal region 160. In this embodiment, distal region 120 includes a threaded region that is multi-lead, and in this particular example is dual-lead, with threads 122a and 122b shown in FIG. 1.

Distal region 120 also includes a porous network of interconnected struts 124 in between the threads, one region of which is labeled in FIG. 1. A porous network of interconnected struts may be referred to herein as a porous lattice. FIG. 1 illustrates an example of a porous network of interconnected struts that may be considered to have a general helical configuration that extends between helical threads of the threaded region, as shown. The helical configuration of the porous lattice may be interrupted by one or more fenestrations and/or flutes where no lattice is present (such as shown in FIG. 1), but the porous lattice may still be considered to have a general helical configuration in these examples.

Distal region 120 is multi-lead (dual-lead in this example), the configuration of which adapts distal region 120 to more securely anchor into dense cancellous sacral bone.

The porous lattices herein may comprise an outer porous network of interconnected struts, an example of which is shown in FIG. 1, and which are described in more detail below. Any of the individual struts herein may also be referred to as a beam. Generally, the porous regions in between the threads preferably have a smooth outer profile to facilitate rotational insertion and proper anchoring. Alternatively stated, it is generally desirable for the porous regions in between threads to avoid having a significantly rough outer surface with exposed strut ends, which may deleteriously damage the adjacent bone and result in less stable anchoring. The porous network may have an irregular configuration of struts, or it may have a regular pattern of struts, or a combination thereof. It is therefore understood that the term lattice as used herein does not require a regular or repeating pattern of struts. Additional exemplary features of porous networks of interconnected struts are described below.

The implant 100 also includes central or intermediate region 140, which is sized and configured (including relative to other implants regions) to be positioned across a SI joint when the implant 100 is delivered laterally across the SI joint. Central region 140 includes a fewer-lead threaded region than distal region 120 and proximal region 160, and in this embodiment is single-lead. In this embodiment thread 142 in central region 140 is considered to continue into distal region 120 as thread 122b, as shown, but in alternative embodiments the central region thread may be considered part of a different thread that does not continue or extend into distal region 120. Thread 122b is considered continuous with thread 142 from distal region 120 into central region 140, even though the thread is interrupted one or more times by fenestrations and fluted regions, which are described in more detail below.

Central region 140 includes a porous network of interconnected struts 144 (which may be referred to here as a lattice), only one region of which is labeled in FIG. 1. Lattice 144, like lattice 124, is disposed between threads in central region 140. The porous lattice 144 may be considered continuous with porous lattice 124 in that they together approximate a generally helical configuration extending from distal region 120 into central region 140, which again is interrupted by one or more fenestrations and fluted region as shown.

The exemplary single-lead design in central region 140 provides for relatively greater axial spacing between threads, compared to, for example, distal region 120. This relatively greater axially spacing creates a greater porous lattice 144 surface area, which is better adapted and configured to facilitate ingrowth and/or ongrowth when central region 140 is implanted across the SI joint. Distal region 120 and central region 140 are examples of regions in which the central region has a relatively greater spacing between threads, which provides for a greater porous surface area between threads.

The elongate body also includes proximal region 160, which includes a threaded region with a greater lead than central region 140. In this example, proximal region 160 includes a threaded region that is dual-lead, as shown. Thread 162a in proximal region 160 is continuous with thread 142 in central region 140, although in alternative embodiments they may not be continuous. It is again understood that the phrase continuous in this context includes one or more interruptions with fluted region and/or fenestrations, as shown in FIG. 1. The multi-lead threaded region in proximal region 160 facilitates strong anchoring in dense iliac bone when implant 100 is implanted laterally across a SI joint.

As mentioned above, implants herein may have a distal region, a central region and a proximal region that are each configured and sized to provide one or more functions based on the anatomical region in which they are positioned after the implant is fully implanted. In some embodiments, any of the distal regions herein (e.g., distal region 120 in FIG. 1) may have a length from 10 mm to 22 mm, for example. This may ensure that the multi-lead region is anchored into dense sacral bone near the mid sacrum. In some embodiments, any of the central regions herein (e.g., central region 140) may have a length from 8 mm to 56 mm. This may ensure that the central region is positioned across the SI joint, with the relatively larger porous surface area extending across the joint to facilitate one or more of ingrowth and/or ongrowth areas. In some embodiments, any of the proximal regions herein (e.g., proximal region 160 in FIG. 1) may have lengths from 6 mm to 10 mm, which can ensure that the multi-lead threaded proximal region is anchored into dense iliac bone. The proximal regions herein with respect to their lengths are not considered to include a proximal end of the elongate body that is free of threads, such as where reference number 104 is pointing in FIG. 1.

Implant body 100 also includes an inner shank or inner member from which the one or more threads and one or more porous lattice regions radially extend. The inner shank may be considered the same or similar to a shank of a screw or other threaded body. The inner shanks herein need not be considered to be continuous structures, and may include one or more breaks or discontinuities therein, such as one or more fenestrations extending therethrough. The inner shank or inner members herein in this context may be considered to include inner surfaces from which one or more threads and one more porous lattice structures extend radially therefrom.

The distal region 120 is tapered towards its distal end, as shown in FIG. 1, a feature of which may be incorporated into any of the implants herein. Distal end 102 in this example also includes sharpened distal end elements, which may be incorporated into any of the implants herein.

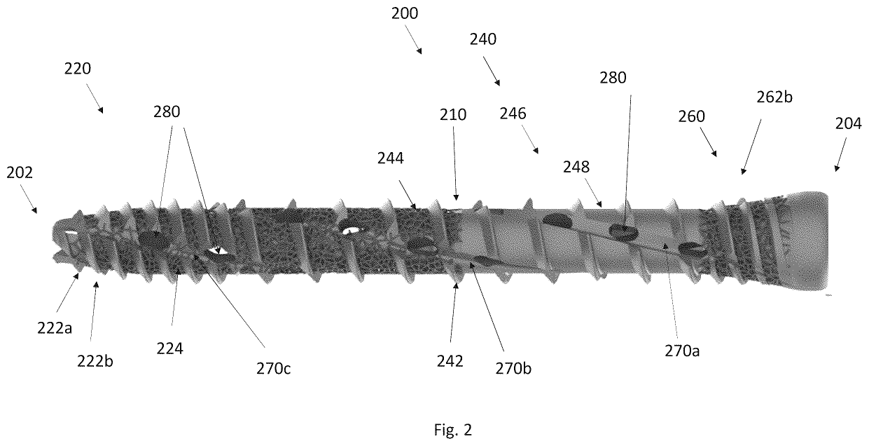

FIG. 2 illustrates an exemplary threaded bone implant 200. Implant 200 may have one or more features of implant 100 in FIG. 1, including features that may be similarly labeled (e.g., 120 and 220). One difference between implant 100 and implant 200 is that implant 200 includes a central region, a proximal portion 246 of which is void or free of a porous network of interconnected struts. Proximal portion 246, which may be considered a solid portion, is disposed at the SI joint when the implant is fully implanted. The proximal portion 246 of the central single threaded region 240 includes inner member or inner shank 248 and a thread radially extending therefrom. In some embodiments, implant 200 may be the same as implant 100 in all other ways. As shown in FIG. 2, shank 248 in proximal portion 246 has the same or substantially the same radial dimension (e.g., diameter) as the porous network of interconnected struts 244 in the distal portion of the central region 240. An exemplary advantage of the proximal portion 246 without the lattice, and the larger diameter shank in proximal portion 246, is that proximal region 246 may be stronger and more fatigue resistant in the region of the larger diameter shank. This may be important on some bone implants with certain dimensions where including a lattice structure along its entire length, including a region across the SI joint, may reduce fatigue strength to an extent that is undesired. For example, in some embodiments, implant 200 may have a major diameter from 9 mm to 11 mm (outer diameter of the thread), such as 10 mm.

As shown in FIG. 2, inner shank 248 has step-up in region 210 in central region 240, where the diameter of the shank increases at the step-up from the distal portion of central region 240 to proximal portion 246 of central region 240. The step-up in shank diameter increases the fatigue strength in the proximal portion 246, which is generally positioned across the joint.

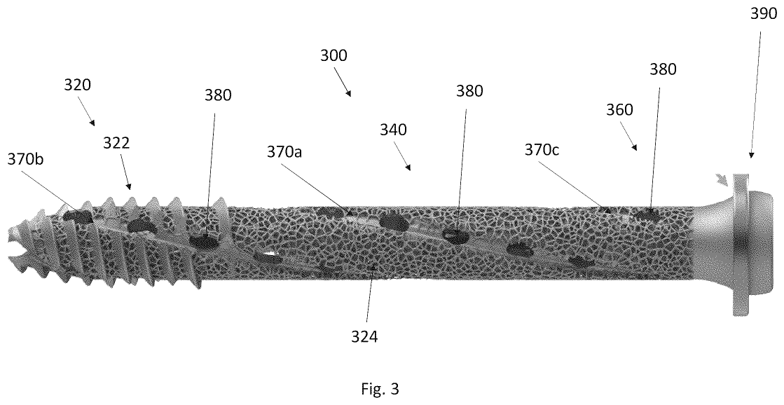

FIGS. 3-6 illustrate exemplary lag threaded implants 300-600, respectively, in which the central regions and proximal regions are not threaded, as shown. The lag implants include a proximal washer, as shown, methods of use of which are generally known for lag implants. In some applications, the threaded lag implants herein may be used for fracture and repair, for example.

FIG. 3 illustrates an exemplary threaded lag implant 300 that includes a distal threaded region 320, a central non-threaded region 340 spaced to be disposed across a SI joint, and a proximal non-threaded region 360. Distal region 340 is multi-lead, and in this embodiment in dual-lead. Implant 300 includes a plurality of helical flutes or fluted region 370 (e.g., 370a, 370b and 370c, as shown). Implant 300 further includes a plurality of fenestrations 380, which may be similar or the same as any of the fenestrations herein. For example, and as shown, each of the helical flutes 370 is aligned with a plurality of fenestrations 380. Fenestrations in the distal region 320 may be larger than fenestrations in the central and/or proximal regions 340 and 360 respectively, such as for the reasons set forth herein.

As shown in FIG. 3, implant 300 includes porous lattice or network of interconnected struts 324, additional exemplary details of which are described herein. Porous lattice 324 may also be considered to have a helical configuration, disposed between both threads 322 and the flutes, as shown. In this example, the porous lattice extends in the distal region 320, the central region 340, and into the proximal region 360. Any of the description herein related to a porous network of interconnected struts may be incorporated into lattice 324. Washer 390 is also shown, and is configured to be disposed about the proximal end of implant 300 and allows for a range of motion between implant 300 and washer 390.

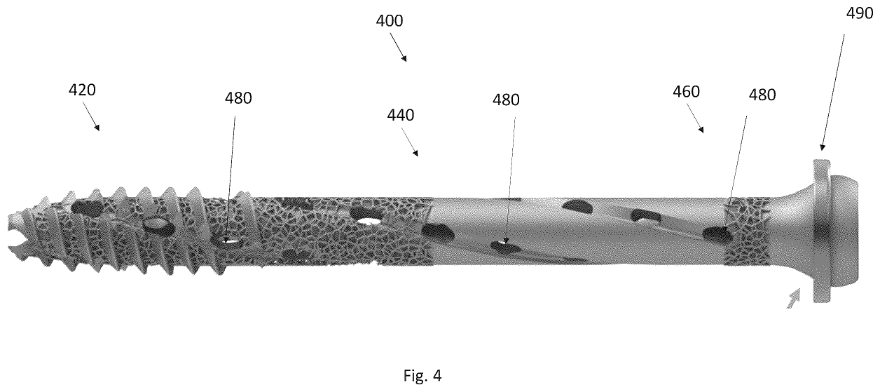

FIG. 4 illustrates implant 400 and illustrates features similar or the same as implant 200 in FIG. 2, in particular a proximal portion of central region 440 that is free of a porous lattice. The relevant description of FIG. 2 with respect to a section free of a porous lattice is incorporated by reference into the description of implant 400 in FIG. 4 for all purposes. An exemplary advantage of the proximal portion of the central region 440 without the lattice as shown, and the larger diameter shank in the proximal portion of central region 440, is that the proximal region may be stronger and more fatigue resistant in the region of the larger diameter shank, for the same reasons set forth herein with respect to FIG. 2.

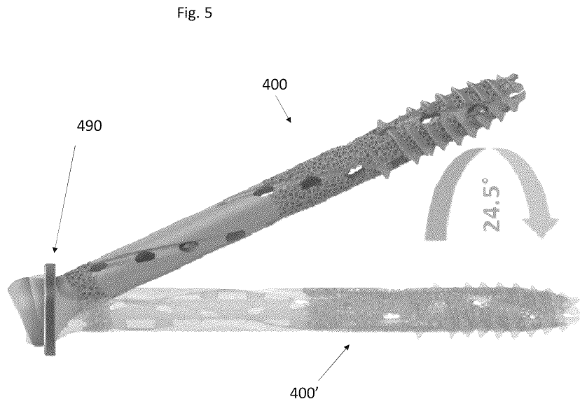

FIG. 5 illustrates implant 400 from FIG. 4, and also illustrates an exemplary angle of rotation and washer 490, with implant 400′ shown to illustrate the exemplary angle of rotation.

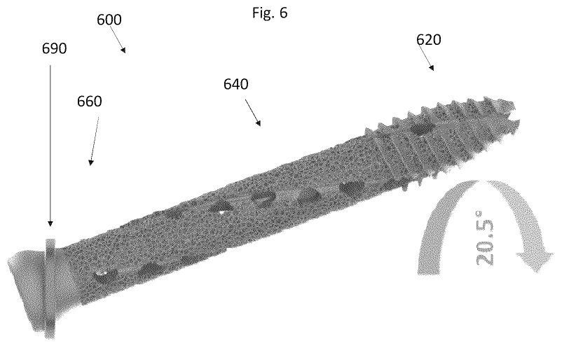

FIG. 6 illustrates implant 600 that may be similar or the same as implant 300 shown in FIG. 3. FIG. 6 illustrates an exemplary angle of rotation and 690. Any suitable description herein related to implant 300 is incorporated by reference into the disclosure of FIG. 6.

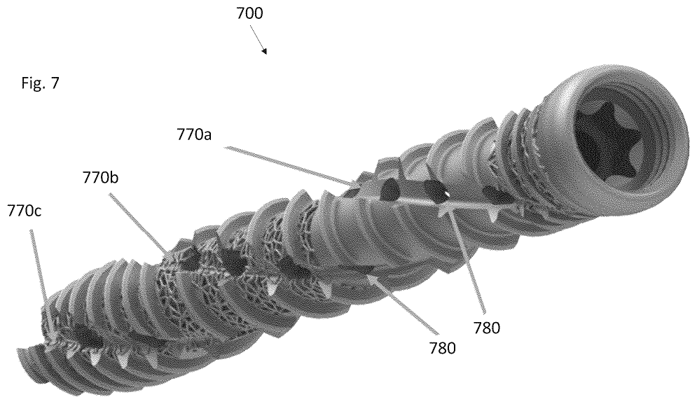

Threaded bone implants herein may include one or more flutes, or fluted regions, examples of which are shown in FIGS. 1-6. FIG. 7 illustrates exemplary threaded bone implant 700, which may include any other suitable feature of any other threaded bone implant described herein. Implant 700 has an elongate body that includes a plurality of helical flutes or fluted regions 770a, 770b, 770c, extending along at least a portion of the length of the elongate body. Similarly, FIG. 2 shows implant 200 including a plurality of helical flutes 270a, 270b and 270c formed therein. Threaded bone implants herein may include three flutes (as shown in the examples of FIGS. 2 and 7), although implants herein may be modified to include more or fewer than three flutes.

Implant 700 also includes fenestrations 780, only two of which are labeled in FIG. 7. Implant 700 is another example of an implant body that includes flutes 770 that are each aligned with a separate plurality of fenestrations 780 formed through the elongate body. Each fluted region in this example includes a separate plurality or set of fenestrations aligned with the respective fluted region, as is shown in FIG. 7.

FIGS. 2 and 7 show exemplary implants that include a plurality of helical flutes, each of which extends from the distal region and into and through the central region, and which may optionally extend in the proximal regions. As shown in FIG. 2, the plurality of helical flutes may extend to a minimal extent into the proximal multi-lead region 260, but the flutes may optionally not extend all the way through proximal regions herein.

As is shown in FIGS. 2 and 7 (but shown in other embodiments herein), the flutes or fluted regions (as well as one or more of the fenestrations) of the implant create an interruption in the one or more threads that extend around the elongate body.

The threaded implants herein may include one or more fenestrations, or relatively larger apertures, extending therethrough. FIG. 2 illustrates a plurality of fenestrations 280 (only two of which are labeled). FIGS. 2 and 7 are examples of threaded implants in which at least one (optionally all) of the fenestrations is aligned, or overlaps with, a fluted region of the implant. FIGS. 2 and 7 each illustrate a plurality of fluted regions of the respective threaded implant, each of which is aligned or overlapped with a plurality of fenestrations. The fenestrations aligned with or overlapping each of the fluted regions are axially spaced apart along the fluted region, and together the fenestrations are disposed in a helical configuration, as shown more clearly in FIG. 7. In FIGS. 2 and 7, for example, there are three sets of helically-oriented fenestrations, each set including a plurality of fenestrations.

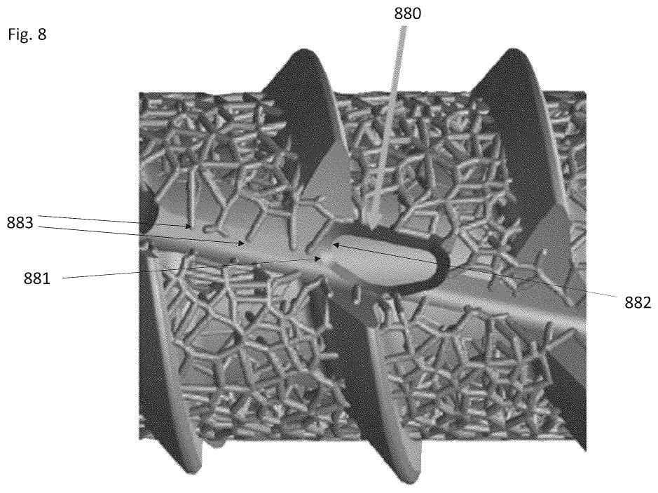

In any of the embodiments herein, any or all of the fenestrations in the implant may have a tapered configuration in the radial direction. FIG. 8 illustrates a single fenestration 880 in a threaded implant elongate body having a tapered configuration between a larger radially outer fenestration opening 881 and a smaller radially inner fenestration opening 882, wherein the difference in opening sizes defines the tapered configuration. This type of taper is referred to herein as a radially inward taper. Any or all of the fenestrations in the threaded implant may be tapered in this manner. Fenestration 880 is also an example of a fenestration aligned with a fluted region, as shown. FIG. 8 is also an example of a continuous thread, as shown, that is interrupted by a fluted region and fenestration 880.

Any of the implants herein may have a plurality of fenestrations, but not all of the implant fenestrations may have the same size or configuration as one or more other fenestrations in the implant. For example, in some embodiments, a distal region of the implant (e.g., distal region 220 in FIG. 2) may not need to have as much fatigue strength as a more proximally disposed region, such as central region 240, or a proximal portion 246 of the central region, which may be implanted across a SI joint. Any of the implants herein may thus have distal regions with one or more fenestrations therein that are larger than one or more fenestrations in at least a portion of the central region that is disposed across the SI joint. The threaded implant central regions may have smaller fenestration so that the implant has more structural material in the region that is disposed across the SI joint. The distal region, which may not need the same fatigue strength, can have more openings, such as in the form of larger fenestrations, without negatively impacting strength of the implant.

Additionally, any of the implants herein may include fenestrations in the distal region that have less pronounced tapers in which there is less of a difference in size or circumferential area between the radially inner opening and the radially outer opening (i.e., a steeper transition between the inner opening and the outer opening). Compared to one or more central region fenestrations, distal region fenestrations may have radially inner openings that are relatively larger than radially inner openings in the central region of the implant.

As described herein, any of the implants herein may include porous regions disposed radially outward from an inner member or shank, wherein the porous regions extend along at least a portion of the threaded implant, including in regions in between the one or more threads. For example, FIG. 1 shows implant 100 that includes porous network of interconnected struts (e.g., 124) in between threads and extending along substantially all of the elongate body. FIG. 2 shows an example of an implant 200 that includes porous regions (e.g., 224) in between threads and extending along at least a distal region of an implant, and in a proximal region of the implant.

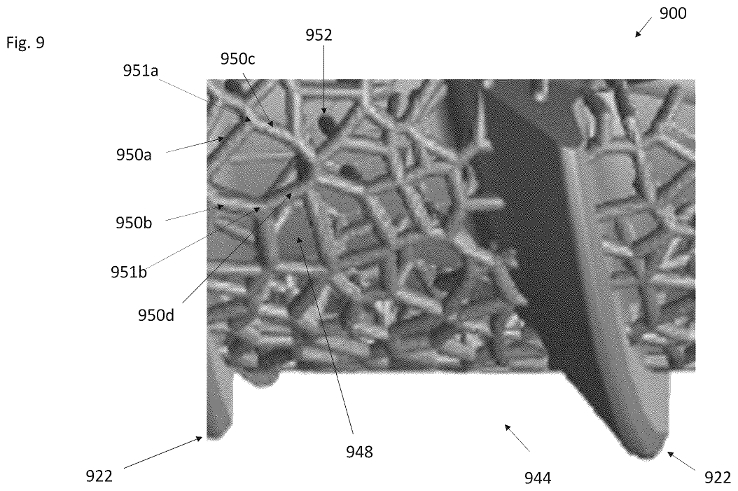

Any of the porous network of interconnected struts herein (e.g., lattice 144 in FIG. 1, porous lattice 244 in FIG. 2) may be a porous network of interconnected struts disposed about the inner shank, an example of which is shown in FIG. 9. With threaded implants such as those described herein, it may be desirable to have the porous network of interconnected struts that are between the threads to rotationally approximate a smooth shank and thereby facilitate a smooth rotational entry into the bone. This can create a minimal amount of resistance and bony damage as the threaded implant is rotated through bone, helping securely anchor the threaded implant into bone. This may be contrasted with porous region that include struts with many free ends that extend radially outward and are not interconnected with other struts as a network. The porous regions herein may be configured as a porous network of interconnected struts that are disposed about an inner shank (e.g., 948), an exemplary highlighted region of which is shown in FIG. 9.

FIG. 9 illustrates a portion of an exemplary implant 900 including thread 922, in between which the implant includes a porous network of interconnected struts 944. Network of interconnected struts 944 includes a plurality of interconnected struts 950 (e.g., 950a, 950b, 950c), only some of which are labeled in FIG. 9 for clarity. The struts 950 are interconnected at connections or nodal locations 951, and only two of which are labeled for clarity—951a and 951b. The connections or nodal locations herein may be the connection of two, three, four, or more individual struts or beams of the porous network of interconnected struts. As set forth above, the porous network of interconnected struts preferably creates a smooth outer surface and may approximate a cylindrical shank (even though the network defines a plurality of pores between the struts), which facilitate a relatively smooth rotation of the implant through bone.

The porous network of interconnected struts has an outer dimension less than a major diameter (diameter of thread(s)) of the at least one helical thread, which is shown in at least FIGS. 8 and 9.

The porous networks of interconnected struts herein may be defined in a variety of ways. For example, the porous network of interconnected struts may be considered to be substantially concentric about a long axis of the elongate body in at least a portion of the porous network of interconnected struts, which is partially shown in the perspective view of FIG. 7. Additionally, the interconnected struts in the porous networks of interconnected struts herein may be considered to have the same radially outermost dimension and concentric about an elongate body long axis. The porous networks of interconnected struts herein may be considered to define a generally circular shape in an end view of the elongate body, which is partially shown in FIG. 7. Additionally, the interconnected struts in the porous networks of interconnected struts herein may be considered to approximate an outer cylindrical profile, even though there are pores defined by the struts, and even though threads may interrupt sections of the outer cylindrical profile. Additionally, the porous network of interconnected struts may be considered to define a generally cylindrical outer profile, even though there are pores defined by the struts, and even though threads may interrupt sections of the generally cylindrical profile. Additionally, the porous networks of interconnected struts may be considered to define a substantially smooth outer surface, even though there are pores defined by the struts. Additionally, any of the porous networks of interconnected struts herein may be considered to include radially outer struts, which are substantially free of strut free ends extending radially outward.

As shown in FIG. 9, the porous lattice may further include a plurality of generally radially extending struts 952 that extend radially outward from the inner shank or inner member 948 and connect to the porous network of interconnected struts. The plurality of radially extending struts 952 generally couple the inner shank 948 to the outer porous network of interconnected struts. Radially extending struts as described in this context (e.g., strut 952) are not necessarily orthogonal, as they may have some radial dimension in addition to some axial dimension.

In any of the embodiments herein, the porous network of interconnected struts includes struts or beams, any of which may have a diameter from 0.175 mm to 0.300 mm.

In any of the embodiments herein, the porous network of interconnected struts may include point spacings from 0.375 mm to 0.525 mm.

In some embodiments herein, such as is shown in FIG. 1, the porous networks of interconnected struts may have or define a general helical configuration extending along the elongate body between the at least one helical thread. Helically extending porous networks herein may have interruptions formed therein and are still considered to have helical configurations.

Any of the porous networks of interconnected struts herein may include one or more end regions including strut free ends (e.g., 883 in FIG. 8), wherein the strut free ends are coupled or extending from a fluted region of the elongate bone implant body, particularly in embodiments in which the threaded implanted is 3D printed. A strut free end in this context refers to a strut end that is not directly connected to another strut, and may be directly connected to another portion of the implant such as an inner shank, a thread or a flute, for example.

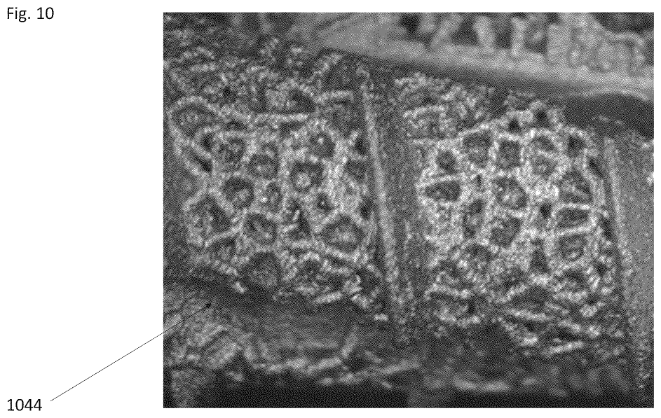

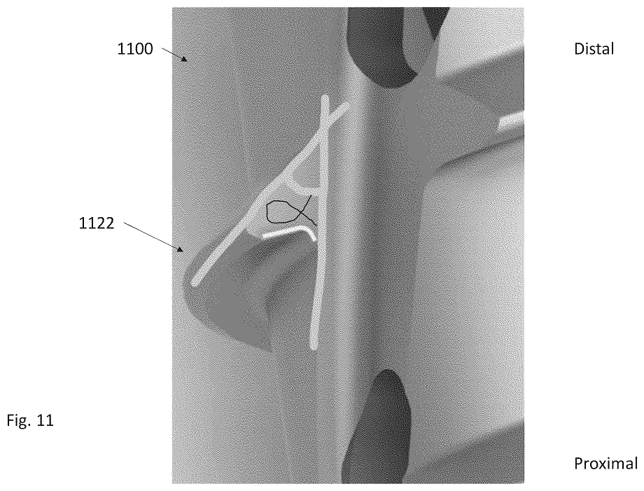

Any the threaded implants herein may be 3D printed, using one or more generally known methods or techniques. FIG. 11 illustrates a portion of an exemplary implant 1100, which may include any of the features of any of the threaded bone implants herein. Relative distal and proximal directions are labeled. Thread 1122 shown in FIG. 11 may be the same or substantially the same as any of the threads shown in the examples of FIGS. 1-10. When threaded bone implants herein, including the threads described herein, are 3D printed with the proximal or head side down, the threads are printed in the orientation shown in FIG. 11. When angle alpha as shown is large enough, the threads may tend to droop proximally (towards the head) during a 3D printing process. For example, in some embodiments, alpha may be greater than 45 degrees, such as from 45 degrees to 75 degrees, such as 45 degrees to 65 degrees. 3D printing some types of threaded bone implants in a head-to-tip direction may thus produce threads that do not have the desired configuration after the printing process.

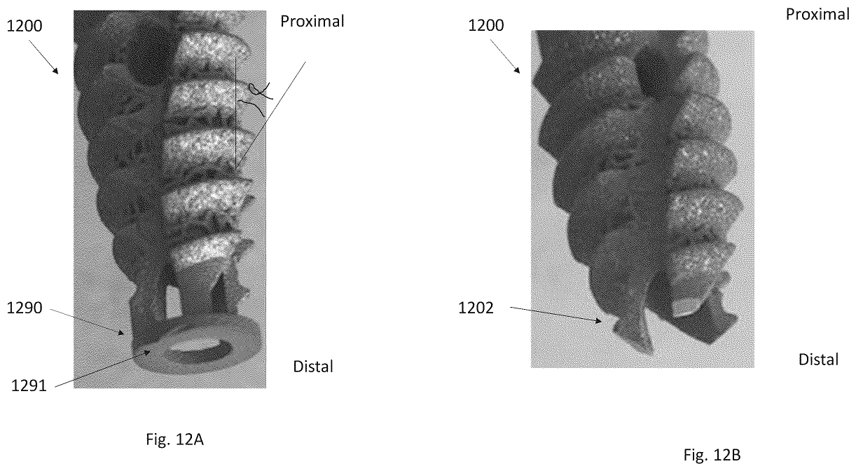

One option to manufacture threaded bone implants with threads that are disposed at certain angles is to print the threaded implants from the tip end (distal end) up to the head end (proximal end), the orientation of which is generally shown in FIG. 12A. Printing in this orientation may, depending on the thread angles, produce threads at angles that are beneficially less likely to droop or sag during the printing process. To print some threaded bone implants, it may be important to have a sturdy base upon which to print the implant upward to maintain a vertical long axis throughout the printing process. FIG. 12A illustrates an exemplary distal portion of 3D printed threaded implant 1200, including a printed sacrificial tip 1290 with a flattened base 1291, which is removed (e.g., machining away) after the printing process to create the finished and optionally sharpened distal tip configuration shown in FIG. 12B. In this example, the sacrificial tip 1290 includes a flattened base 1291 that provides a sturdy base upon which the implant may be printed up towards the head or proximal region. Printing in this orientation with an optional sacrificial sturdy base may allow for 3D printing some threaded implants that would be challenging to print if attempts were made to print from the proximal head upward to a distal tip end.

One aspect of the disclosure is a method of a method of 3D printing a threaded bone implant (such as any of the threaded implants herein). The method may include printing a threaded bone implant from a distal end to a proximal end. The method may include printing an inner shank, printing at least one helical thread extending along at least a portion of the threaded bone implant and extending from the inner shank. The method may also include printing a porous network of interconnected struts about the inner shank, about a long axis of the elongate bone implant body, and between at least a section of the at least one helical thread, where the porous network of interconnected struts has an outer dimension less than a major diameter of the at least one helical thread. The method may include printing the porous network of interconnected struts to be substantially concentric about a long axis of the elongate body in at least a portion of the porous network of interconnected struts. The method may include printing the porous network of interconnected struts to have a general helical configuration extending along the elongate body between the at least one helical thread and about the inner shank. The method may include printing the porous network of interconnected struts to have the same radially outermost dimension and concentric about an elongate body long axis. The method may include printing strut ends that are disposed within and coupled to fluted regions of the implant. The method may include printing the porous network of interconnected struts to define a substantially smooth radially outer surface that approximate a cylindrical profile.

One aspect of the disclosure is a method of printing a threaded bone implant. The method may include 3D printing a sacrificial distal tip and printing a threaded bone implant above the sacrificial tip. The method may include removing the sacrificial tip (e.g., machining it away) and forming a distal end, optionally sharpened, on the distal end of the bone implant after removing the sacrificial tip.

It is understood that features of one or more embodiments herein may be integrated with one or more other embodiments herein unless the disclosure indicates to the contrary.

1.-15. (canceled)

16. A threaded bone implant (“implant”), comprising:
an elongate body (“body”) extending from a proximal end to a distal end, the elongate body including,
one or more threads in a tapered distal section of the body, in a central section of the body, and in a proximal section of the body, the one or more threads axially spaced further apart in the central section than in the tapered distal section and in the proximal section,
a network of interconnected struts that define a plurality of openings between the interconnected struts to provide for one or more of bony on-growth, ingrowth or through-growth, the network of interconnected struts having a helical configuration between the one or more threads in the central section, and
the one or more threads in the central section having a plurality of thread interruptions that together are helically arranged in a direction that is different than a direction of the helical configuration of the network of interconnected struts.

17. The implant of claim 16, wherein the tapered distal section is multi-lead, the central section is single-lead, and the proximal section is multi-lead.

18. The implant of claim 16, wherein a first thread of the one or more threads is continuous from within the proximal central, into the central section, and into the distal tapered section, the first thread including at least some of the plurality of thread interruption in the central section that are helically arranged.

19. The implant of claim 16, wherein the network of interconnected struts has a greater surface area in the central section than in the tapered distal section and then in the proximal section.

20. The implant of claim 16, wherein the one or more threads and the network of interconnected struts are integral, formed together with an additive manufacturing process.

21. The implant of claim 16, wherein the elongate body has a central lumen.

22. The implant of claim 16, wherein the network of interconnected struts has a continuous helical configuration from within the distal section, into the central section, and into the proximal section.

23. The implant of claim 16, wherein the network of interconnected struts has a smooth outer profile.

24. The implant of claim 16, wherein the network of interconnected struts is free of strut free ends extending radially outward.

25. The implant of claim 16, wherein the network of interconnected struts approximates an outer cylindrical profile to facilitate a smooth rotation of the implant through bone.

26. A threaded bone implant (“implant”), comprising:
an elongate body (“body”) extending from a proximal end to a distal end, the elongate body including,
one or more threads in a tapered distal section of the body, a central section of the body, and a proximal section of the body, the one or more threads axially spaced further apart in the central section than in the tapered distal section and in the proximal section,
a network of interconnected struts the define a plurality of openings between the interconnected struts to provide for one or more of bony on-growth, ingrowth or through-growth, the network of interconnected struts having a helical configuration between the one or threads in the central section, and
wherein the tapered distal section is multi-lead, the central section is single-lead, and the proximal section is multi-lead.

27. The implant of claim 26, wherein a first thread of the one or more threads is continuous from within the proximal central, into the central section, and into the distal tapered section.

28. The implant of claim 27, wherein the first thread includes a plurality of thread interruptions in the central section, the plurality of thread interruptions having a helical arrangement.

29. The implant of claim 26, wherein the network of interconnected struts has a greater surface area in the central section than in the tapered distal section and then in the proximal section.

30. The implant of claim 26, wherein the one or more threads and the network of interconnected struts are integral, formed together with an additive manufacturing process.

31. The implant of claim 26, wherein the elongate body has a central lumen.

32. The implant of claim 26, wherein the network of interconnected struts has a continuous helical configuration from within the distal section, into the central section, and into the proximal section.

33. The implant of claim 26, wherein the network of interconnected struts is free of strut free ends extending radially outward.

34. The implant of claim 26, wherein the network of interconnected struts approximates an outer cylindrical profile to facilitate a smooth rotation of the implant through bone.
